(19) United States
(12) Patent Application Publication
(10) Pub. No.: US 2023/0121963 A1
(43) Pub. Date: Apr. 20, 2023

(54) BREATHING APPARATUS TO ELIMINATE AIRBORNE INFECTIONS

(71) Applicant: Novacc Clean Air, LLC, Silver Creek, GA (US)

(72) Inventor: Kenneth Wright, Silver Creek, GA (US)

(21) Appl. No.: 18/046,964

(22) Filed: Oct. 17, 2022

Related U.S. Application Data
(60) Provisional application No. 63/257,098, filed on Oct. 18, 2021.

(51) Int. Cl. A62B 7/12; A61L 9/16; A62B 9/00; A62B 9/02

(52) U.S. Cl. CPC A62B 7/12; A61L 9/16; A62B 9/003; A62B 9/02; A62B 9/006; A61M 16/1075

(57) ABSTRACT

A breathing apparatus may include a heater configured to kill harmful organisms in air. The apparatus may include a vest, including a heating chamber disposed on the vest and a cooling chamber disposed on the vest. The heating chamber may include a heat source, and the heat source may heat air disposed within the heating chamber to a predetermined temperature. The heating chamber and the cooling chamber may be in fluid communication. The apparatus may include a breathing tube, including a first end and a second end. The first end may connect to the cooling chamber. The apparatus may include a breathing mask. The second end of the breathing tube may connect to the breathing mask. The cooling chamber and the breathing mask may be in fluid communication via the breathing tube.

### CROSS-REFERENCES TO RELATED APPLICATIONS

This application claims priority to U.S. Provisional Patent Application No. 63/257,098, entitled “BREATHING APPARATUS TO ELIMINATE AIRBORNE INFECTIONS,” filed on Oct. 18, 2021, which is pending. The entirety of this application is incorporated by reference.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### STATEMENT REGARDING FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### BACKGROUND OF THE DISCLOSURE

The present disclosure generally relates to preventing infections, and more particularly to a breathing apparatus for eliminating airborne infections.

Current approaches for treating and preventing infections mainly involve defensive approaches (e.g., washing hands, social distancing, wearing masks, and administering vaccines). Currently, an individual can utilize every one of these defensive measures and still become infected with an airborne illness, require hospitalization, or die.

The primary cause of transmission for many airborne illness is aerosol transmission. Social distancing addresses droplet transmission (10-100 microns) being exhaled by a contaminated person (e.g., through sneezing or coughing). Washing hands only addresses surface contamination and transmission. Aerosols (5-10 microns) can remain airborne for up to 18 hours. Most humans do not wear personal protective equipment (PPE) or masks that block particles this small. Wearing masks that fail to treat or kill the infectious agent may only serve to recirculate it in an asymptomatic patient.

One of the most prevalent airborne illnesses today is the SARS-CoV-2 virus (“COVID”). When inhaled, COVID will incubate and take around 10-12 hours to penetrate a cell and begin to replicate itself. Approximately 4-6 people out of every 100 who become infected with COVID are asymptomatic. Thus, many people can be infected without knowing they are infected and contagious. Furthermore, cases of COVID have exploded in places with mask mandates because droplet transmission is not the leading cause of transmission.

What is needed, then, is a breathing apparatus for eliminating airborne illnesses.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

One aspect of the disclosure is a breathing apparatus. The breathing apparatus may include a heater configured to kill harmful organisms in air. The apparatus may include a vest, including a heating chamber disposed on the vest and a cooling chamber disposed on the vest. The heating chamber may include a heat source, and the heat source may heat air disposed within the heating chamber to a predetermined temperature. The heating chamber and the cooling chamber may be in fluid communication. The apparatus may include a breathing tube, including a first end and a second end. The first end may connect to the cooling chamber. The apparatus may include a breathing mask. The second end of the breathing tube may connect to the breathing mask. The cooling chamber and the breathing mask may be in fluid communication via the breathing tube.

Another aspect of the disclosure includes another breathing apparatus including a heater configured to kill harmful organisms in air. The apparatus may include an air intake. The apparatus may include a heating chamber. The apparatus may include an air intake channel disposed between the air intake and the heating chamber. The air intake and the heating chamber may be in fluid communication via the air intake channel. The apparatus may include a heat source disposed in the heating chamber. The apparatus may include a cooling chamber in fluid communication with the heating chamber. The apparatus may include an air channel connected to the cooling chamber. A first one-way valve may be disposed between the heating chamber and the cooling chamber. A second one-way valve may be disposed on an end of the air channel and disposed opposite the cooling chamber.

Another aspect of the disclosure includes a system. The system may include a vehicle. The system may include a breathing apparatus disposed in the vehicle. The breathing apparatus may include a heater, including an interior space. The interior space may include a heating chamber and a cooling chamber. A heat source may be disposed within the heating chamber. The breathing apparatus may include a breathing tube, including a first end and a second end disposed opposite the first end. The first end of the breathing tube may connect to the heater. The second end of the breathing tube may selectably coupleable to a breathing mask. The heat source may heat air disposed within the interior space to a predetermined temperature.

Numerous other objects, advantages and features of the present disclosure will be readily apparent to those of skill in the art upon a review of the following drawings and description of various embodiments.

### BRIEF DESCRIPTION OF THE DRAWINGS

FIG. 1 is a front view illustrating one embodiment of a breathing apparatus disposed on a subject.

FIG. 2 is a perspective view illustrating another embodiment of a breathing apparatus disposed on a subject.

FIG. 3 is a schematic diagram illustrating another embodiment of a breathing apparatus.

FIG. 4A is a front view illustrating one embodiment of a breathing apparatus in a vest configuration disposed on a subject.

FIG. 4B is a back view illustrating another embodiment of the breathing apparatus in the vest configuration disposed on the subject.

### DETAILED DESCRIPTION

While the making and using of various embodiments of the present disclosure are discussed in detail below, it should be appreciated that the present disclosure provides many applicable inventive concepts that are embodied in a wide variety of specific contexts. The specific embodiments discussed herein are merely illustrative of specific ways to make and use the disclosure and do not delimit the scope of the disclosure. Those of ordinary skill in the art will recognize numerous equivalents to the specific apparatus and methods described herein. Such equivalents are considered to be within the scope of this disclosure and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

Reference throughout this specification to “one embodiment,” “an embodiment,” “another embodiment,” or similar language means that a particular feature, structure, or characteristic described in connection with the embodiment is included in at least one embodiment. Thus, appearances of the phrases “in one embodiment,” “in an embodiment,” “in some embodiments,” and similar language throughout this specification may, but do not necessarily, all refer to the same embodiment, but mean “one or more but not necessarily all embodiments” unless expressly specified otherwise.

The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. As used herein, the term “a,” “an,” or “the” means “one or more” unless otherwise specified. The term “or” means “and/or” unless otherwise specified.

A better approach to preventing the spread of airborne illness would be a more “offensive” approach by killing the illness. Such approach may include diluting an infected subject with fresh air instead of recirculating infected air.

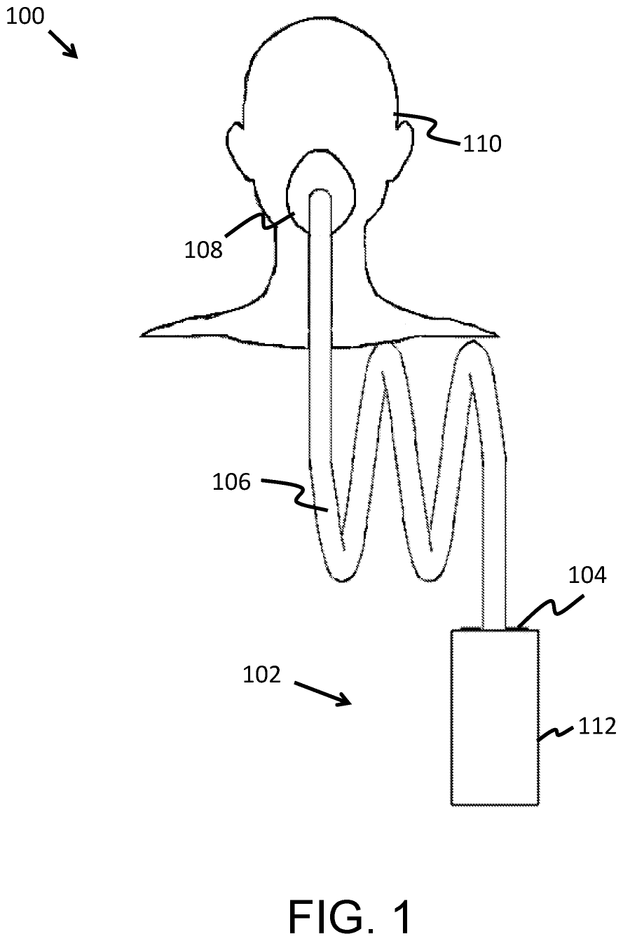

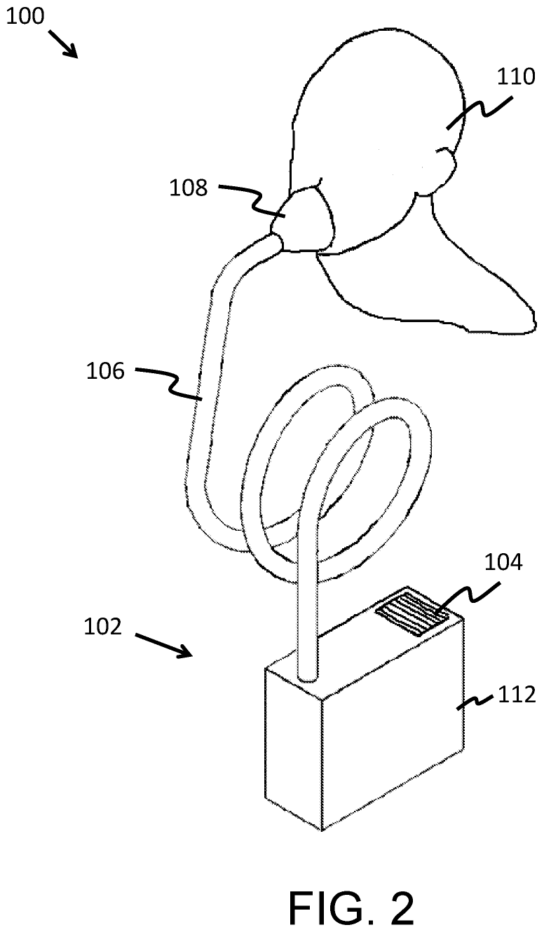

FIG. 1 and FIG. 2 depict various views of one embodiment of an apparatus 100. The apparatus 100 may include a breather apparatus. The apparatus 100 may be based upon the scientific principle of utilizing heat to kill viruses, bacteria, or other potentially harmful organisms. The apparatus 100 may include a heater 102. The heater 102 may include an air intake 104. The apparatus 100 may include a breathing tube 106. The apparatus 100 may include a breathing mask 108. In some embodiments, the breathing mask 108 may be wearable by a subject 110. The subject 110 may include a human.

The heater 102 may receive air via the air intake 104. The heater 102 may heat the received air to a temperature sufficient to kill viruses, bacteria, or other harmful organisms. The subject 110 may inhale the air that was heated by the heater 102 from the heater 102 through the breathing tube 106 and the breathing mask 108. By doing this, the subject 110 may inhale air that is substantially free from viruses, bacteria, and other harmful organisms that cannot live above the heated temperature. This may allow the subject 110 to breathe purified, prevent the subject 110 from becoming sick, or prevent the subject 110 from spreading disease.

Further details about the apparatus 100 will now be disclosed. In one embodiment, the apparatus 100 may include the heater 102. The heater 102 may include components for receiving air from the heater's 102 surrounding environment, heating the received air to purify the air, and sending the purified air to the breathing tube 106 and breathing mask 108 to be inhaled by the subject 110. In one embodiment, the heater 102 may include an interior space for holding air. The heater 102 may heat the air in the interior space to kill viruses, bacteria, or other potentially harmful organisms or particles. In some embodiments, the interior space of the heater 102 may include separate interior spaces: one to hold air received from the heater's 102 surrounding environment, and another to receive air exhaled from the subject 110. In some embodiments, the heater 102 may ensure that these two types of air do not mix.

In some embodiments, the heater 102 may heat air inside the interior space of the heater 102. The heater 102 may include a heat source that may perform the heating. The heat source may heat the air disposed within the interior space to a predetermined temperature. The predetermined temperature may include approximately 162 degrees Fahrenheit (72 degrees Celsius). In some embodiments, the predetermined temperature may include some other value. The predetermined temperature may range between 100 and 120 degrees, 120 and 140 degrees, 140 and 160 degrees, 160 and 180 degrees, 180 and 200 degrees, 200 and 220 degrees Fahrenheit, or some other range of temperatures. The temperature may include a temperature sufficient to kill the COVID-19 virus in 1 second. The apparatus 100 may be applicable to other viruses (e.g., H1N1, SARS, MERS, Influenza, etc.). The heating temperature of the heater 102 and the amount of time that air may spend in the heater 102 may be adjustable. In one or more embodiments, the heater 102 may include an air output. The air output may include one or more components that may expel air from the heater 102. The expelled air may include air that the subject 110 has exhaled.

In some embodiments, the heater 102 may heat air exhaled from the subject 110. Heating the exhaled air may kill live viral particles from the subject 110, which may reduce a viral load and may prevent the subject 110 from spreading an infection into the atmosphere. In some embodiments, the heater 102 may include a fan. The fan may provide airflow through the heater 102. The fan may intake air from the air intake 104. The fan may propel air through the breathing tube 106. The fan may intake air from the breathing tube 106. The fan may expel air from the heater 102 to an air output.

In one embodiment, the heater 102 may include a power source. The power source may provide power to the heater 102 to run one or more components of the heater 102. The heater 102 may use the power to consistently heat the incoming air. The heater 102 heating the air in the heater 102 may consume less electricity than running a hairdryer. The power source may provide a constant heating of the heat source to warm the air to the required degree range. Further details regarding one or more interior components of the heater 102 are provided below.

In certain embodiments, the heater 102 may include an exterior case 112. The exterior case 112 may surround and enclose the interior components of the heater 102. The air intake 104 may be disposed on the exterior case 112. The breathing tube 106 may extend from the exterior case 112. The exterior case 112 may include metal, plastic, or some other rigid material. The exterior case 112 may be lightweight to aid in the portability of the heater 102. In some embodiments, the heater 102 may include a handle. The handle may disposed on the exterior case 112, and the subject 110 may carry the heater 102 by the handle. The handle may include a rigid handle, a handle that pivots to allow the handle to lie against the exterior case 112, or some other handle.

In one embodiment, the heater 102 may include an air intake 104. The air intake 104 may include an aperture that may allow the heater 102 to receive air from the surrounding environment. The air intake 104 may include a vent, a filter, a grate, or some other component that may allow air to pass into the heater 102 but may prevent other larger objects from entering the heater 102. The air intake 104 may be disposed on a side of the heater 102, the top of the heater 102, the bottom of the heater 102, or some other area of the heater 102. The air intake 104 may intake air in response to the subject 110 breathing in, which may create a vacuum effect and may cause the air in the heater 102 to be vacuumed into the breathing tube 106 and, eventually, into the subject 110. The air intake 104 may intake air in response to other functionality of the heater 102, for example, a fan disposed in the heater 102 drawing air into the heater 102.

In some embodiments, the apparatus 100 may include the breathing tube 106. The breathing tube 106 may allow air to flow from the heater 102 to the breathing mask 108. The breathing tube 106 may include a first end. The breathing tube 106 may include a second end disposed opposite the first end. The first end may be disposed at the heater 102. The second end may be disposed on the breathing mask 108. The breathing tube 106 may include a flexible material. The flexible material may include an air-tight material. The flexible material may include a plastic (such as a polymer such as polyvinyl chloride (PVC)), metal, or some other material. The breathing tube 106 may include different lengths. The length of the breathing tube 106 may be adjustable. For example, a portion of the breathing tube 106 may be retractable into the heater 102.

In some embodiments, the breathing tube 106 may allow the air to cool before entering human lungs. In some embodiments, an interior space of the heater 102 may include a cooling section or portion that may cool the purified air before propagating it through the breathing tube 106. In other embodiments, the cooling section may be located elsewhere on the apparatus 100.

In one embodiment, the apparatus 100 may include the breathing mask 108. The breathing mask 108 may allow the subject 110 to breath in purified air from the heater 102 and may at least partially prevent the subject 110 from breathing unpurified air from the surrounding environment. The breathing mask 108 may include a mask that is selectably coupleable to the subject's 110 face and that has an aperture for receiving air from the breathing tube 106. The breathing mask 108 may be selectably coupleable to the subject's 110 face such that the breathing mask 108 covers the subject's 110 nose or mouth. In some embodiments, the breathing mask 108 may be formable to different sizes and shapes of a human face of the subject 110. The breathing mask 108 may seal out all incoming or outgoing air. The breathing mask 108 may include a zipper, hook-and-loop fastener, a strap, a tie, or some other type of disposal mechanism. The disposal mechanism may be adjustable to adjust for different sizes, circumferences, or other portions of the subject's 110 head. In one or more embodiments, exhaled air from the subject 110 may propagate from the breathing mask 108, through the breathing tube 106, and to the heater 102.

In certain embodiments, the breathing mask 108 may include a mask that is selectively coupleable to the breathing tube 106. The second end of the breathing tube 106 may selectably couple to the breathing mask 108. The subject 110 or some other user may detach the breathing mask 108 from the breathing tube 106 and may attach a different breathing mask 108 to the breathing tube 106. The coupling between the breathing mask 108 and the breathing tube 106 may include an air-tight coupling that may prevent air, viral particles, and other particles from escaping from the breathing mask 108 or the breathing tube 106 when coupled. In some embodiments, the breathing tube 106 may selectively couple to the heater 102. The subject 110 or some other user may detach the breathing tube 106 from the heater 102 and may attach a different breathing tube 106. The coupling between the breathing tube 106 and the heater 102 may include an air-tight coupling. In one or more embodiments, the selectably coupleable breathing mask 108 or breathing tube 106 may allow a subjects 110 to bring their own breathing masks 108 or breathing tubes 106 to a facility or vehicle (e.g., an airplane, train, bus, boat, etc.) and plug in the breathing mask 108 or breathing tube 106 to the heater 102 provided at the facility or vehicle.

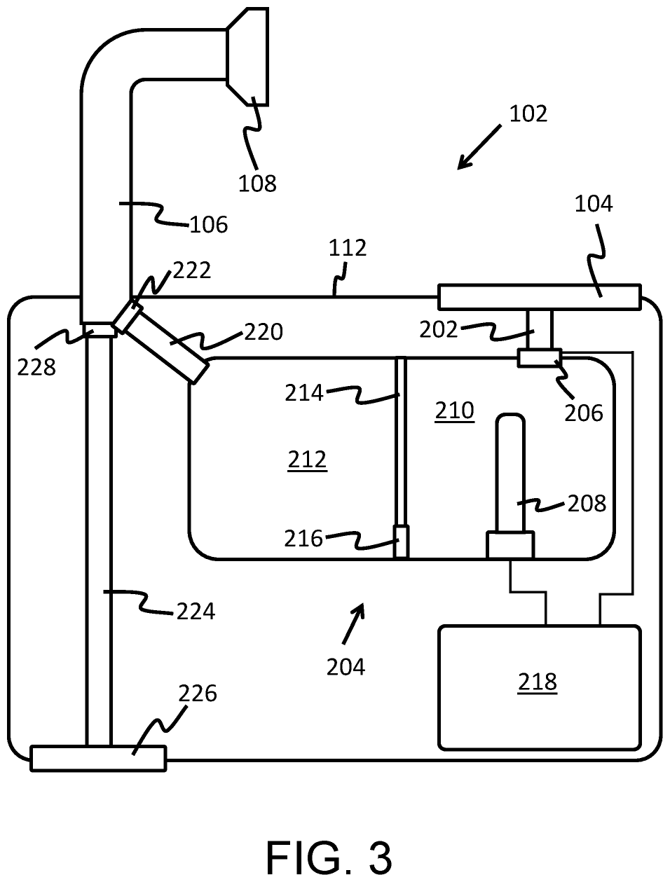

FIG. 3 depicts one embodiment of the apparatus 100. The apparatus 100 may include the heater 102, which may include an air intake 104. The heater 102 may include an air intake channel 202, an interior space 204, a fan 206, or a heat source 208. The interior space 204 may include a heating chamber 210 and a cooling chamber 212. The interior space 204 may include a divider 214 that may divide the two chambers 210, 212 and a first one-way valve. The heater 102 may include a power source 218. The heater 102 may include an air channel, a second one-way valve 222, an air output channel 224, an air exhaust port 226, or a third one-way valve 228.

In some embodiments, the heater 102 may include the air intake channel 202. The air intake channel 202 may transfer air from the air intake 104 to the interior space 204 of the heater 102. The air intake channel 202 may include a channel that includes a metal, a plastic, or some other material. The air intake channel 202 may include a variety of lengths or widths.

In some embodiments, the interior space 204 may include an area disposed inside the heater 102 that may hold or store air. The interior space 204 may include various sizes and capacities for air. The interior space 204 may include an insulating material that surrounds the interior space 204. The insulating material may help prevent heat from escaping the interior space 204 and into surrounding components of the heater 102.

In some embodiments, the heater 102 may include a fan 206. The fan 206 may cause air disposed outside the heater 102 to move into the interior space 204. The fan 206 may be disposed in the air intake channel 202, between the air intake channel 202 and the interior space 204, near the air intake 104, or in some other location.

In some embodiments, the heater 102 may include the heat source 208. The heat source 208 may heat the air in the interior space 204 in order to kill viral particles, bacteria, or other harmful or undesired particles. The heat source 208 may include a heating coil, heat exchanger, a heating element, a fiber, graphene, or some other heat source. The heat source 208 may be disposed in various locations in the interior space 204. For example, the interior space 204 may be divided into a heating chamber 210 and a cooling chamber 212. The heat source 208 may be disposed in a central portion of the heating chamber 210, near the exit of the air intake channel 202, near the fan 206, or in some other location.

In some embodiments, the interior space 204 may include a divider 214 that may divide the heating chamber 210 from the cooling chamber 212. In some embodiments, the heating chamber 210 and the cooling chamber 212 may be formed from two different and separate interior spaces 204. The heating chamber 210 and the cooling chamber 212 may be in fluid communication. In some embodiments, a one-way valve 216 may be disposed between the heating chamber 210 and the cooling chamber 212. The one-way valve 216 may allow air to flow from the heating chamber 210 to the cooling chamber 212, but not from the cooling chamber 212 to the heating chamber 210. The heating chamber 210 may hold heated air, and the cooling chamber 212 may allow the heated air that flows into the cooling chamber 212 to reach a safe temperature for inhalation by the subject 110. In some embodiments, the cooling chamber 212 may include a cooling element to cool the air within the cooling chamber 212.

In some embodiments, the heater 102 may include a power source 218. The power source 218 may provide power to the heat source 208, the fan 206, a cooling element, or other components of the heater 102. The power source 218 may include a battery. The battery may include a rechargeable battery. The battery may include a lithium ion, lithium polymer, or metal hydride battery. The power source 218 may include an electric cord, a solar panel, a hand crank, or some other type of power source. The power source 218 may be rechargeable or swappable.

In one embodiment, the heater 102 may include an integrated circuit, application-specific integrated circuit (ASIC), or other circuitry (not shown in FIG. 3). The circuitry may receive power from the power source 218. The circuitry may receive input from one or more sensors disposed on the heater 102, process the input, and, based on that processing, perform one or more operations that may control one or more functions of the hardware of the heater 102. For example, the heating chamber 210 may include a temperature sensor electronically connected to the circuitry. The circuitry may receive a temperature reading from the temperature sensor that may indicate the temperature of the heating chamber 210. In response to the temperature of the heating chamber 210 being below a threshold temperature (e.g., 162 degrees Fahrenheit, as discussed above), the circuitry may activate the heat source 208. In response to the temperature sensor indicating the heating chamber 210 has reached a temperature above a threshold temperature, the circuitry may deactivate the heat source 208.

In one embodiment, the heater 102 may include an air channel 220. The air channel 220 may be disposed between the interior space 204 (e.g., as shown in FIG. 3, the cooling chamber 212) and the breathing tube 106 such that the interior space 204 and the breathing tube 106 are in fluid communication. In response to the subject 110 breathing in, a vacuum effect may be created and the air may flow from the interior space 204, through the air channel 220, into the breathing tube 106, and out through the breathing mask 108. In some embodiments, the heater 102 may include a second one-way valve 222. The second one-way valve 222 may allow air to travel from the air channel 220 through the second one-way valve 222 and into the breathing tube 106, but may prevent air from traveling from the breathing tube 106 into the air channel 220 (and, thus, into the interior space 204). The first end of the breathing tube 106 may be selectably coupleable to the cooling chamber 212 via the air channel 220.

In some embodiments, the heater 102 may include an air output channel 224. The air output channel 224 may be disposed between the breathing tube 106 and an air exhaust port 226 such that the breathing tube 106 and the air exhaust port 226 are in fluid communication. In response to the subject 110 breathing out, the air exhaled from the subject 110 may flow from the breathing tube 106 through the air output channel 224 and out of the heater 102 through the air exhaust port 226. A third one-way valve 228 may allow air to travel from the breathing tube 106 through the third one-way valve 228 and into the air output channel 224, but may prevent air from traveling from the air output channel 224 into the breathing tube 106.

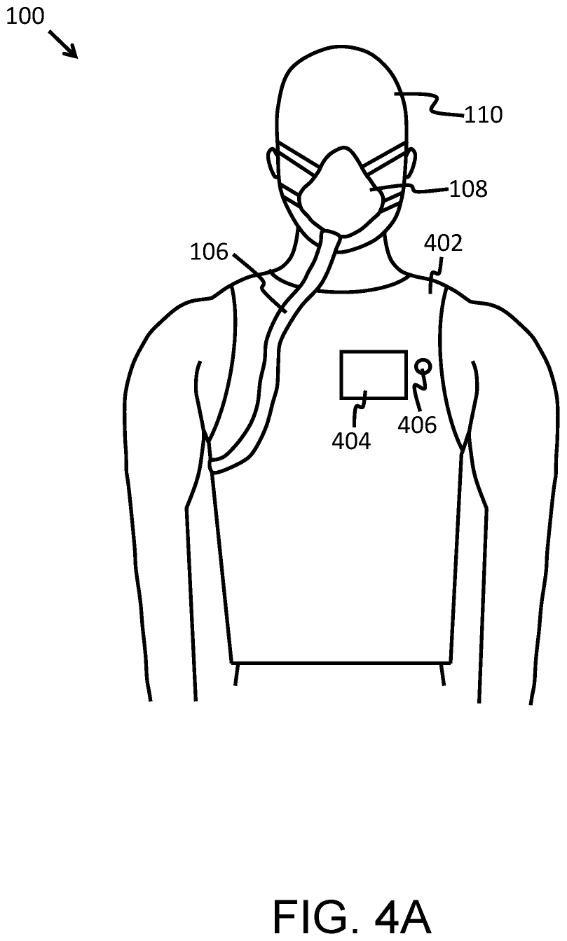

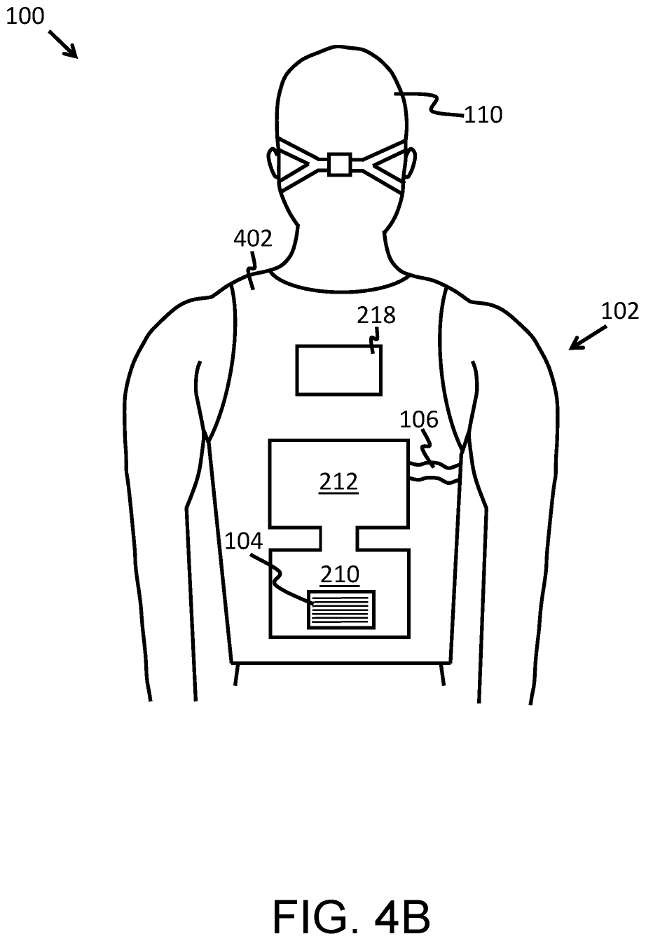

FIG. 4A and FIG. 4B depict another embodiment of the apparatus 100. In one embodiment, the apparatus 100 may include a vest 402. The vest 402 may include a lightweight utility vest. The subject 110 may wear the vest 402. As depicted in FIG. 4A, the front side of the vest 402 may include a display 404. The display 404 may show information about the apparatus 100, such as a current temperature of the interior space 204, the heating chamber 210, or the cooling chamber 212, a desired temperature for the interior space 204, the heating chamber 210, or the cooling chamber 212, an amount of charge remaining in the power source 218, or other information. The display 404 may include a screen, such as a liquid crystal display (LCD), a light-emitting diode (LED) display, a segment display, a touchscreen, or some other type of display. The display 404 may be in electric or electronic communication with the power source 218. The display 404 may be mounted on the vest 402. The display 404 may be separate from the vest 402 but may be electrically connected to the vest 402 (e.g., via one or more wires of the vest 402, such as electrical wires supplying power to the display 404 via the power source 218). In other embodiments, the display 404 may include a wireless display in data communication with a circuitry of the vest 402. The display 404 may include the display of a smartphone or other computing device in wired or wireless data communication with the circuitry.

In one or more embodiments, the vest 402 may include a light 406. The light 406 may include a LED or some other type of light. The light 406 may include a red-green-blue (RGB) light. The light 406 may display a certain color, a certain brightness level, or a certain blinking pattern in order to convey information about the apparatus 100. For example, the light 406 may display green in response to the heater 102 working properly or in response to the power source 218 having a charge above a threshold charge. The light 406 may display yellow in response to the heater 102 encountering an error or in response to the power source 218 having a charge below a threshold charge (e.g., below 30% power). The light 406 may display red in response to the heater 102 not being able to function or in response to the power source 218 having below a second threshold charge (e.g., below 10% power). In some embodiments, the display 404 or the light 406 may be included in other embodiments of the apparatus 100, such as embodiments shown and discussed above in relation to FIGS. 1-3.

As shown in FIG. 4B, the back side of the vest 402 may include certain components of the heater 102. For example, the vest 402 may include the heating chamber 210 disposed on the vest 402, the cooling chamber 212 disposed on the vest 402, the air intake 104, or the power source 218 disposed on the vest 402. The vest 402 may include other components of the apparatus 100 discussed above in relation to FIGS. 1-3. As can be seen in FIGS. 4A-B, the breathing tube 106 may couple to the cooling chamber 212 and may wrap around the vest 402 and to the breathing mask 108. Certain portions of the breathing tube 106 may couple to the vest 402 in order to not obstruct the subject 110. For example, the vest 402 may include loops, adhesives, hooks, or other components that may secure portions of the breathing tube 106 to the vest 402.

In one embodiment, the apparatus 100 may include a filter. The filter may kill the infectious agent. The filter may include peroxide, disinfectant, bleach, or some other type of filter. The filter may be disposed in the heater 102 (e.g., in the air intake channel 202, in the air channel 220, or in the air output channel 224), in the breathing tube 106, in the breathing mask 108, or at some other location on the apparatus 100.

In one or more embodiments, the apparatus 100 may include a user interface. The user interface may receive input from the subject 110 or another user of the apparatus 100, and in response, the apparatus 100 may perform one or more functions based on the input. In some embodiments, the user interface may electronically connect to the circuitry of the heater 102. In one embodiment, the user interface may include one or more buttons, knobs, dials, touchscreens, or other user input components. These user input components may adjust the temperature to which the heat source 208 heats the air in the interior space 204.

The apparatus 100 disclosed herein provides several advantages over the prior attempted solutions. For example, in some embodiments, the apparatus 100 may be lightweight, comfortable, weather-resistant, compact, inexpensive, or easily sanitized. The apparatus 100 may not restrict vision, hearing, nor the ability to work, drive, or sleep. In some embodiments, the heater 102 may be portable, for example, within a backpack or side bag (e.g., a messenger bag or purse) or worn on a vest 402. The heater 102 may be able to be wheeled about on a cart or other wheeled device. In some embodiments, one or more components of the apparatus 100 may be heat-resistant, chemical-resistant, lightweight, waterproof, or impermeable to viral particle transmission.

In some embodiments, the apparatus 100 may provide the option for those deemed at greater risk at becoming infected (e.g., doctors, nurses, healthcare workers, nursing home workers, assisted living centers, workers with large groups in confined surroundings, schools/colleges, etc.) to protect themselves from such infection. The breathing mask 108 may only cover the subject's 110 nose and mouth, which may provide more comfort, vision, or other advantages over full-face shields or masks.

Furthermore, other methods of killing potentially harmful airborne organisms, such as chemical sterilization, lasers, or ionization radiation may be too resource-intensive or may pose greater safety risks than the heating disclosed herein. The heating may also be faster than ultraviolet light, which requires a substantial amount of time to kill the potentially harmful organisms. The power source 218 in the form of a battery also provides advantages over solutions with power cords, such as improved mobility and range and the absence of cables that can obstruct or entangle. Additionally, the apparatus 100 may be modular. Certain components of the apparatus 100 may be coupleable to other components, allowing for easy replacement of these components. Such modular components may include the breathing mask 108, the breathing tube 106, the power source 218, the interior space 204, the heating chamber 210, the cooling chamber 212, the heating element 208, or other components.

Thus, although there have been described particular embodiments of the present disclosure of a new and useful BREATHING APPARATUS TO ELIMINATE AIRBORNE INFECTIONS, it is not intended that such references be construed as limitations upon the scope of this disclosure.

What is claimed is:

1. A breathing apparatus including a heater configured to kill harmful organisms in air, comprising:
a vest, including
a heating chamber disposed on the vest, and
a cooling chamber disposed on the vest, wherein
the heating chamber includes a heat source,
the heat source heats air disposed within the heating chamber to a predetermined temperature, and
the heating chamber and the cooling chamber are in fluid communication;
a breathing tube, including
a first end, wherein the first end connects to the cooling chamber, and
a second end disposed opposite the first end; and
a breathing mask,
wherein
the second end of the breathing tube connects to the breathing mask, and
the cooling chamber and the breathing mask are in fluid communication via the breathing tube.

2. The apparatus of claim 1, wherein the predetermined temperature comprises at least 162 degrees Fahrenheit.

3. The apparatus of claim 1, wherein the heat source comprises at least one of:
a heating coil;
a heat exchanger;
a heating element;
a fiber; or
graphene.

4. The apparatus of claim 1, wherein:
the vest further comprises a power source connected to the heat source; and
the power source supplies power to the heat source.

5. The apparatus of claim 4, wherein the power source comprises at least one of:
a battery;
an electric cord;
a solar panel; or
a hand crank.

6. The apparatus of claim 4, further comprising a display mounted on the vest.

7. The apparatus of claim 6, wherein the display is in electronic communication with the power source.

8. The apparatus of claim 1, wherein at least one of:
the first end of the breathing tube is selectably coupleable to the cooling chamber; or
the second end of the breathing tube is selectably coupleable to the breathing mask.

9. The apparatus of claim 1, wherein the heating chamber and the cooling chamber are disposed on a back side of the vest.

10. A breathing apparatus including a heater configured to kill harmful organisms in air, comprising:
an air intake;
a heating chamber;
an air intake channel disposed between the air intake and the heating chamber, wherein the air intake and the heating chamber are in fluid communication via the air intake channel;
a heat source disposed in the heating chamber;
a cooling chamber in fluid communication with the heating chamber; and
an air channel connected to the cooling chamber,
wherein
a first one-way valve is disposed between the heating chamber and the cooling chamber, and
a second one-way valve is disposed on an end of the air channel and disposed opposite the cooling chamber.

11. The apparatus of claim 10, wherein a breathing tube is selectably coupleable to the heater.

12. The apparatus of claim 11, wherein:
the breathing tube includes a first end and a second end disposed opposite the first end;
the first end is selectably coupleable to the heater; and
the second end includes a breathing mask.

13. The apparatus of claim 10, further comprising a breathing tube disposed on the heater, wherein the breathing tube includes a first end and a second end disposed opposite the first end, and wherein the first end of the breathing tube disposed on the heater.

14. The apparatus of claim 13, wherein a breathing mask is selectably coupleable to the second end of the breathing tube.

15. The apparatus of claim 10, wherein the heat source heats air disposed within the heating chamber to a predetermined temperature.

16. The apparatus of claim 15, wherein the predetermined temperature comprises at least 162 degrees Fahrenheit.

17. A system, comprising:
a vehicle; and
a breathing apparatus disposed in the vehicle, the breathing apparatus including
a heater, including
an interior space, including a heating chamber and a cooling chamber, and
a heat source disposed within the heating chamber, and
a breathing tube, including
a first end, and
a second end disposed opposite the first end; and
wherein
the first end of the breathing tube connects to the heater,
the second end of the breathing tube is selectably coupleable to a breathing mask, and
the heat source heats air disposed within the interior space to a predetermined temperature.

18. The system of claim 17, wherein the vehicle comprises at least one of:
an airplane;
a train;
a bus; or
a boat.

19. The system of claim 17, wherein the predetermined temperature comprises at least 162 degrees Fahrenheit.

20. The system of claim 17, wherein the heat source comprises at least one of:
a heating coil;
a heat exchanger;
a heating element;
a fiber; or
graphene.
